(19) United States
(12) Patent Application Publication
(10) Pub. No.: US 2023/0290506 A1
(43) Pub. Date: Sep. 14, 2023

(54) SYSTEMS AND METHODS FOR RAPIDLY SCREENING FOR SIGNS AND SYMPTOMS OF DISORDERS

(71) Applicants: Rehabilitation Institute of Chicago D/B/A Shirley Ryan AbilityLab, Chicago, IL (US); Northwestern University, Evanston, IL (US)

(72) Inventors: Arun Jarayaman, River Forest, IL (US); Luca Lonini, Chicago, IL (US); Chandrasekaran Jayaraman, Seattle, WA (US); Olivia Botonis, Chicago, IL (US); Sung Yul Shin, Glenview, IL (US); Nicholas Shawen, Chicago, IL (US); Chaithanya Krishna Mummidisetty, Sammamish, WA (US); Sophia T. Jenz, Chicago, IL (US); Michael Fanton, Chicago, IL (US)

(21) Appl. No.: 18/006,120

(22) PCT Filed: Jul. 22, 2021
(86) PCT No.: PCT/US2021/042846

§ 371 Date: Jan. 19, 2023

Related U.S. Application Data
(60) Provisional application No. 63/054,866, filed on Jul. 22, 2020.

(51) Int. Cl. G16H 50/20; G16H 50/30; G06N 20/00; G16H 10/60; G16H 50/70; G16H 50/80; A61B 5/0205

(52) U.S. Cl. CPC G16H 50/20; G16H 50/30; G06N 20/00; G16H 10/60; G16H 50/70; G16H 50/80; A61B 5/02055

(57) ABSTRACT

Systems and methods for rapid screening of signs and symptoms associated with disorders are disclosed. A processor or processing element in operable communication with one or more sensors is configured to detect physiological and movement changes associated with a disorder based on signals derived from sensor data generated by the one or more sensors as a user performs a set of predetermined scripted activities.

### CROSS REFERENCE TO RELATED APPLICATIONS

The present PCT patent application claims the benefit of provisional patent application No. 63/054,866 filed on Jul. 22, 2020, which is hereby incorporated by reference to its entirety.

### FIELD

The present disclosure generally relates to disease screening mechanisms; and includes a system and associated method for rapidly screening for signs and symptoms of disorders including COVID-19.

### BACKGROUND

The year 2020 ushered in the Coronavirus disease 2019 (COVID-19) pandemic, the likes of which the modern world had not recently experienced. Scientists around the globe have been working on developing various forms of a vaccine and treatment. In the meantime, there is an urgent need for technology and decision systems to quickly detect whether an individual is exhibiting signs or symptoms similar to those of COVID-19 infection in order to prevent the further spread of this disease and changes to signs and symptoms in response to a drug or other intervention.

It is with these observations in mind, among others, that various aspects of the present disclosure were conceived and developed.

### SUMMARY

In some examples, the present disclosure takes the form of a non-invasive method predicting a disorder diagnosis that does not require presence of a clinician during operation, comprising the steps of: accessing by a processor of a plurality of processing elements screening data generated from a sensor system positioned along an individual of a plurality of individuals for screening the individual for a disorder as the individual performs a predetermined sequence of activities, the predetermined sequence of activities including at least one exertion configured for predicting a presence of the disorder; conducting signal processing by the processor from raw sensor information of the screening data to derive a plurality of signals from each activity of the predetermined sequence of activities, the plurality of signals from an activity collectively predictive for detecting the presence of the disorder; and computing by the processor an output defining a probability measure of risk of a positive diagnosis of the disorder attributable to the individual by applying the plurality of signals defined by the screening data to a machine learning model, parameters of the machine learning model configured, based on the plurality of signals, to maximize a probability of detecting the disorder.

The method may further include the step of configuring the machine learning model, by: accessing by at least one of the plurality of processing elements one or more training datasets, each of the one or more training datasets generated from an implementation of the sensor system positioned along a sample individual of the plurality of individuals as the sample individual performs the predetermined sequence of activities; and conducting signal processing by the processor for each of the one or more training datasets to derive a plurality of sample signals from one or more activities of the predetermined sequence of activities, wherein the machine learning model is trained and configured based on the plurality of sample signals.

The method may further include the step of training the machine learning model by conducting feature extraction by the processor to extract feature values for each of the plurality of sample signals that quantify statistical properties for one or more activities of the predetermined sequence of activities.

In some examples, the present disclosure takes the form of a system (or device) including at least a sensor system positioned along an individual of a plurality of individuals, and a processor in operable communication with the sensor system. By the present example, the processor of the system accesses screening data from the sensor system for screening the individual for a disorder as the individual performs a predetermined sequence of activities, the predetermined sequence of activities including at least one exertion configured for predicting a presence of the disorder. In addition, the processor conducts signal processing from raw sensor information of the screening data to derive a plurality of signals from each activity of the predetermined sequence of activities, the plurality of signals from an activity collectively predictive for detecting the presence of the disorder. The processor further computes an output defining a probability measure of risk of a positive diagnosis of the disorder attributable to the individual by applying the plurality of signals defined by the screening data to a machine learning model. The parameters of the machine learning model are configured, based on the plurality of signals, to maximize a probability of detecting the disorder.

In some examples, the sensor system of the subject system example includes a first sensor positioned along a chest of the individual to monitor movement and gait patterns, respiratory dynamics, and heart dynamics of the individual, and a second sensor positioned along a finger of the individual including a PPG sensing device. The first sensor measures acceleration, ECG, and a first temperature, and the second sensor measures blood-oxygen and a second temperature. The sensor system may include a motion sensor defining an accelerometer and a photopletysmography (PPG) sensor, such that the plurality of signals includes mechano-acoustic signals recorded by the accelerometer and blood oxygen levels recorded by the PPG sensor. The disorder being screened may be a COVID-19 infection. In this example, the plurality of signals includes a heart signal, and a change in heart signal between activities in the predetermined sequence of activities is extracted by the processor as a feature for the machine learning model, and the plurality of signals further includes an acceleration signal indicative of a respiration rate of the individual.

In some examples, the present disclosure takes the form of tangible, non-transitory, computer-readable media having instructions encoded thereon, such that a processor executing the instructions is operable to access screening data from a sensor system for screening an individual for a disorder as the individual performs a predetermined sequence of activities, the predetermined sequence of activities including at least one exertion configured for predicting a presence of the disorder. In addition, the processor executing the instructions is operable to conduct signal processing from raw sensor information of the screening data to derive a plurality of signals from each activity of the predetermined sequence of activities, the plurality of signals from an activity collectively predictive for detecting the presence of the disorder. The processor executing the instructions is further operable to compute an output defining a probability measure of risk of a positive diagnosis of the disorder attributable to the individual by applying the plurality of signals defined by the screening data to a machine learning model. The parameters of the machine learning model are configured, based on the plurality of signals, to maximize a probability of detecting the disorder.

In the subject example, the machine learning model may be preconfigured or trained in the manner described herein, such that the model as applied is suitable for predicting a diagnosis of a disorder.

### BRIEF DESCRIPTION OF THE DRAWINGS

FIG. 1A is a simplified block diagram of a system including a plurality of tangible devices and components for rapid screening of signs and symptoms associated with disorders.

FIG. 1B is a possible architecture supported by or otherwise related to the system of FIG. 1A for rapid screening of signs and symptoms associated with disorders.

FIG. 2 is an illustration demonstrating one embodiment of the system and/or architecture described herein, along with signal types and a model approach associated with the present disclosure.

FIG. 3A is an illustration depicting an example of prompting an individual or user of the system for a standardized activity protocol and data collection process for testing, training, or screening.

FIG. 3B is an illustration of an outline example snapshot or predetermined sequence of activities and indicates possible sensing modalities for the activities, signal features extracted for a machine learning model, and an example implementation of the model as a classifier for detecting COVID or another disorder.

FIG. 3C is a graph illustrating signal data derived from one or more sensors during various activities or stages of the predetermined sequence of activities over time.

FIG. 4A is an illustration and/or data flow diagram indicating data flow to compute signal features and exemplary Machine Learning (ML) model architecture.

FIG. 4B is another illustration and/or data flow diagram indicating data flow to compute signal features and exemplary Machine Learning (ML) model architecture.

FIG. 5A is a simplified block diagram illustrating an example method of the present disclosure for configuring or training a model for rapid screening of signs and symptoms associated with disorders.

FIG. 5B is a simplified block diagram illustrating another method for implementing the model of FIG. 5B for rapid screening of signs and symptoms associated with disorders.

FIG. 6A is a confusion matrix indicating a considerable performance of the model described herein for differentiating between COVID negative and COVID positive patients.

FIG. 6B is a graph illustrating a receiver operating characteristic (ROC) indicating strong predicative performance of the model described herein.

FIG. 6C is an illustration showing classification scores associated with one implementation example of the model.

FIG. 6D is a graph showing features used to differentiate between COVID positive diagnosis participants and COVID negative diagnosis participants.

FIG. 7 is a simplified block diagram of an exemplary embodiment of the sensor system described herein.

FIG. 8 is a simplified block diagram of an exemplary computing device for effectuating various functions of the present disclosure.

Corresponding reference characters indicate corresponding elements among the view of the drawings. The headings used in the figures do not limit the scope of the claims.

### DETAILED DESCRIPTION

Aspects of the present disclosure relate to examples of a system and variations of a customized method to screen individuals for suspect respiratory, cough, oxygen saturation, cardiac, and movement symptoms indicative of various disorders and in some examples to predict a diagnosis of a disorder. The examples provided in the present application are directed toward detection of symptoms related to COVID-19 infection, but those skilled in the art will recognize the same method/s could be modified to specifically detect symptoms of other disorders including but not limited to infections by other viral pathogens, bacterial pathogens, asthma, and COPD. In contrast to recent attempts of detecting COVID-19 based on continuous monitoring through wearable sensors in which the aim is to uncover general trends in the signals, the novel approach of the present disclosure uses a “snapshot” of sensor data generated during a predetermined sequence of activities to detect physiological and movement changes associated with the disease, among other features described herein.

The method may be used as a diagnostic biomarker, and is based on data including one or more of physiological, motion and mechano-acoustic (a.k.a. sensor modalities) datasets recorded with one or more wireless wearable sensors applied on the user, while the user performs a scripted sequence of activities. The sensors can be placed on the user's chest, throat, wrist or finger, and may be connected wirelessly to an application running on a mobile device (phone or tablet), which is used to manage the devices and transfer the recorded data. The mobile application guides the individual through the scripted sequence of activities and records the sensor data associated to each performed activity. An artificial intelligence and/or machine learning (AI/ML) model is trained with the data derived from the sensors to detect the physiological changes associated with these activities, and outputs a probability measure of risk of infection compatible with COVID-19 symptoms. One feature of the method is that the scripted procedure can be completed in a short period of time by the user, and does not require the presence of trained personnel to be executed, thus adhering to the social distancing guidelines recommended during the pandemic. The ML algorithm may run on the user's mobile device; and anonymized data may also be uploaded to a HIPAA compliant cloud server where the data can further be processed and/or can be stored for further analysis.

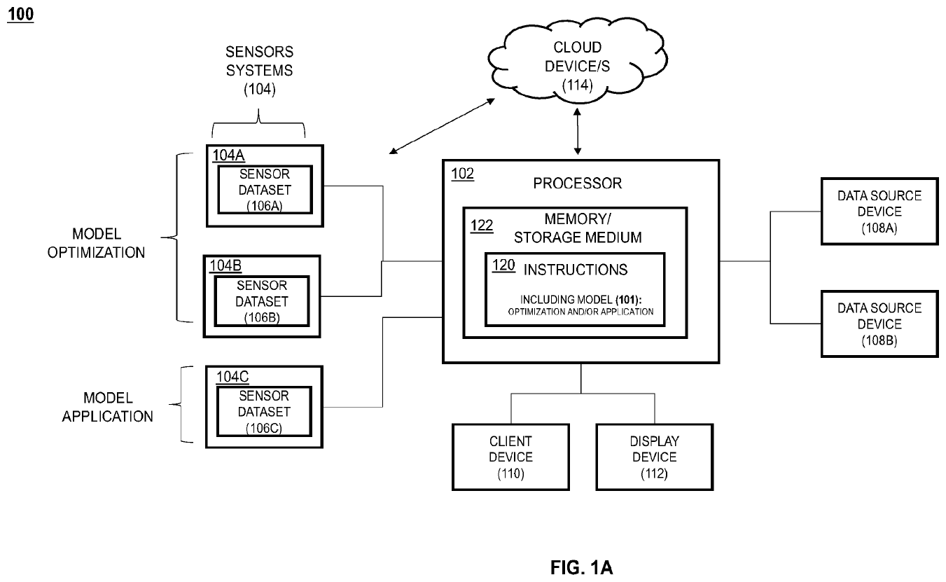

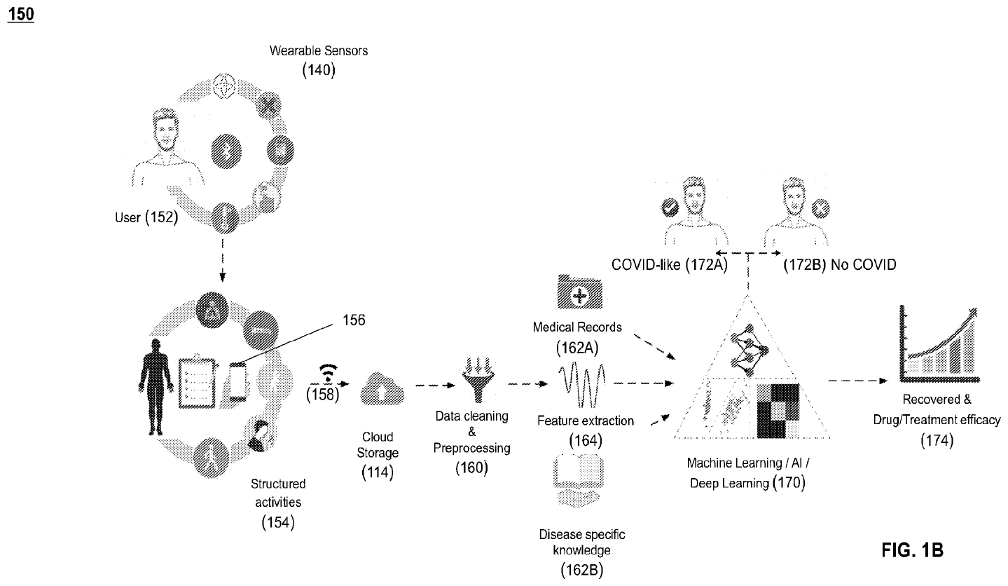

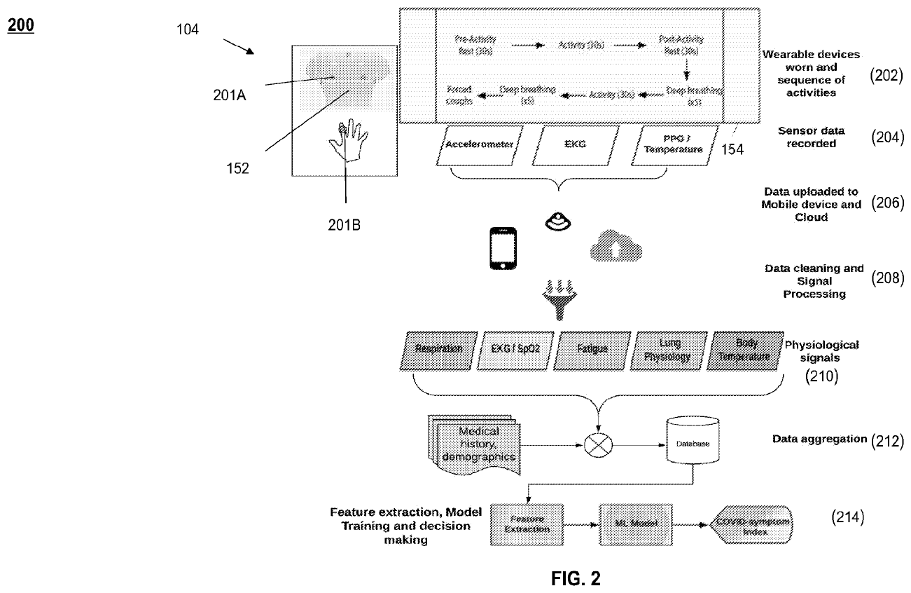

FIG. 1A illustrates a general system 100 of devices and other components for rapid screening of signs and symptoms associated with disorders utilizing an artificial intelligence or machine learning (ML) model 101, and FIG. 1B and FIG. 2 illustrate an example implementation or architecture 150 of the system 100 and indicate various possible features and functions thereof. In particular, the system 100 includes at least a processor 102 that executes the model 101, and a plurality of sensors systems 104 including one or more sensors that generate a plurality of sensor datasets 106 accessible by the processor 102. As shown, the sensor systems 104 may include one or more implementations of a sensor system 104, denoted as sensor system 104A and sensor system 1048 for producing respectively sensor dataset 106A and sensor dataset 106B when positioned along a testing or sample individual as the individual performs a predetermined sequence of activities. Signal features from the sensor dataset 106A and sensor dataset 106B may then be used as inputs to the model 101 for optimization, configuration, and/or training thereof. Sensor system 104C denotes another implementation of the sensor systems 104 that may be positioned along an individual for screening the individual for a disorder. In general, signal features from the sensor dataset 106C generated by the sensor system 104C as the individual performs the same predetermined sequence of activities can be fed to the model 101 as trained to output a probability measure that predicts a diagnosis of a disorder for the individual being screened, as further described herein.

The system 100 may further include at least one data source device 108 shown by non-limiting example as data source device 108A and data source device 108B for optionally providing additional inputs to the model 101. In some examples, aspects of the system 100 and methods described herein may be provided to or intergrated with a client device 110 (e.g., mobile device) and/or a display device 112 in operable communication with the processor 102 for providing feedback. Further, examples of the system 100 may include one or more devices of a cloud 114 which may define cloud computing, storage, processing, infrastructure, and the like. The processor 102 may be a processing element of the cloud 114, and/or leverage processing of computing elements of the cloud 114 in various examples described herein. The components of the system 100 are shown as an example, are non-limiting with respect to form, type, and number and the system 100 may include other related components and variations. While the present inventive concept is described primarily as an implementation of the system, it should be appreciated that the inventive concept may also take the form of a device, and/or a tangible, non-transitory, computer-readable media having instructions encoded thereon and executable by a processor, and any number of methods related to embodiments of the system described herein.

As further shown in FIG. 1B, the processor 102 is configured with instructions 120 stored in memory 122 or some computer-readable medium in operable communication with the processor 102. In general, the instructions 120 define implementation and optimization of the model 101, and may define various functions, algorithms, or methods and the like for interpreting the sensor datasets 106 using artificial intelligence, and optionally using information from the data source devices 108 for rapid screening of signs and symptoms associated with disorders as further described herein. The instructions 120 may be implemented as code and/or machine-executable instructions executable by the processor 102 that may represent one or more of a procedure, a function, a subprogram, a program, a routine, a subroutine, a module, an object, a software package, a class, or any combination of instructions, data structures, or program statements, and the like. In other words, the instructions 120 and functions described herein may be implemented by hardware, software, firmware, middleware, microcode, hardware description languages, or any combination thereof. When implemented in software, firmware, middleware or microcode, the program code or code segments to perform the necessary tasks (e.g., a computer-program product) may be stored in a computer-readable or machine-readable medium (e.g., the memory 122), and the processor 102 performs the tasks defined by the code.

Referring to FIG. 1B and FIG. 2, a general example architecture 150 for rapid screening of signs and symptoms associated with disorders is shown. In general under the architecture 150, sensor data defining one or more of the sensor datasets 106 (FIG. 1A) is generated by at least one sensor system 104 as a testing individual 152 performs one or more of a predetermined sequence of activities (demonstrated as “structured activities” 154 in FIG. 1B). The testing individual 152 may be prompted by a mobile device 156 to perform the predetermined sequence of activities described herein to assess a possible disorder. The sensor data 106 is then fed to storage of the cloud 114 or the processor 102 directly via a wired or wireless communication protocol 158 for processing. Namely, the sensor data 106 is cleansed, filtered, and/or processed, and signals or signal features are derived from the sensor data 106 which are provided as inputs to the model 101. The processor 102, applying the model 101 which includes at least some form of artificial intelligence such as a machine learning or deep learning, determines a diagnosis or disease state for the disorder such as positive or negative (indicated by example in FIG. 1B as positive for “COVID-like” 172A or negative shown as “No-COVID”). Recovery and Drug/Treatment efficacy 174 can then be assessed. Optionally, the model 101 may be supplemented with information from one or more data source devices 108 (providing e.g., medical records 162A and/or disease specific knowledge 162B). In some examples, the processor 102 conducts feature extraction 164 to provide feature values to the model 101.

FIG. 2 shows one example of data flow stages of a process 200 using the system 100 and architecture 150 and using one particular example of the sensor system 104. As indicated, in some examples, the sensor system 104 may include at least one of a first sensor 201A positioned along an upper body or chest of the individual 152, and at least one of a second sensor 201B positioned along a finger of the individual 152 during a first stage 202 of the process 200. During a second stage 204 of the process 200, sensor data is recorded as the individual 152 performs the predetermined sequence of activities 154. As previously indicated, the sensor data may be uploaded to a mobile device, the processor 102, or to a cloud device 114 in stage 206 of process 200 for processing. In stage 208 of process 200, some processing element such as the processor 102 conducts signal processing to derive physiological signals (210) from the sensor data generated by the first sensor 201A and the second sensor 201B during each activity of the predetermined sequence of activities 154. The physiological signals 210 are then used to define or optimize parameters of the model 101. Optionally, as indicated, features may be extracted from the physiological signals for training of the model 101 and the feature values may be aggregated across signals and activities in stages 212 and 214 for model training and decision making.

In some specific examples, the sensor data 106 is generated by one or more of the sensor systems 104 in view of at least one exertion activity of the predetermined sequence of activities performed by the individual 152. In some embodiments, data collection involves high resolution (varying sampling rates between 100 Hz to 2 kHz) sensor modalities capable of measuring body motions and physiological parameters, including EKG and temperature, as well as mechano-acoustic signals, such as cough, breathing, and heart sounds; which may be implemented along or proximate to the user (sensor system 104 may include wearable sensors, as shown in FIG. 1B). For instance, in one such example, a sensor system 104 may take the form of a wireless wearable device configured for high resolution (sampling rate >1 kHz) with the first sensor 201A comprising one or more motion sensors (accelerometer or gyroscope) worn on the upper chest/throat region, and the second sensor 201B comprising a photopletysmography (PPG) sensor worn on the finger to measure changes in blood flow/perfusion. The method of the present disclosure is not limited to a specific sensor. Rather, any sensor that can collect the requisite data may be used. Examples of such sensors are disclosed in PCT Application Serial No. PCT/US2019/018318, filed Feb. 15, 2019, and U.S. patent application Ser. No. 16/190,958, filed Nov. 14, 2018.

After placing the sensors on the body, the user performs the predetermined sequence of activities 154 (also referred to herein as the protocol). Various embodiments or variations of the predetermined sequence of activities 154 can be administered depending on the need of expert clinicians. As one example, the predetermined sequence of activities includes the following activities (sensor data 106 generated during each activity): 30 seconds of breathing normally (e.g., at rest), walking for 30 seconds, 30 seconds of resting, five deep breaths, 30 seconds of walking, five deep breaths, and five forced coughs.

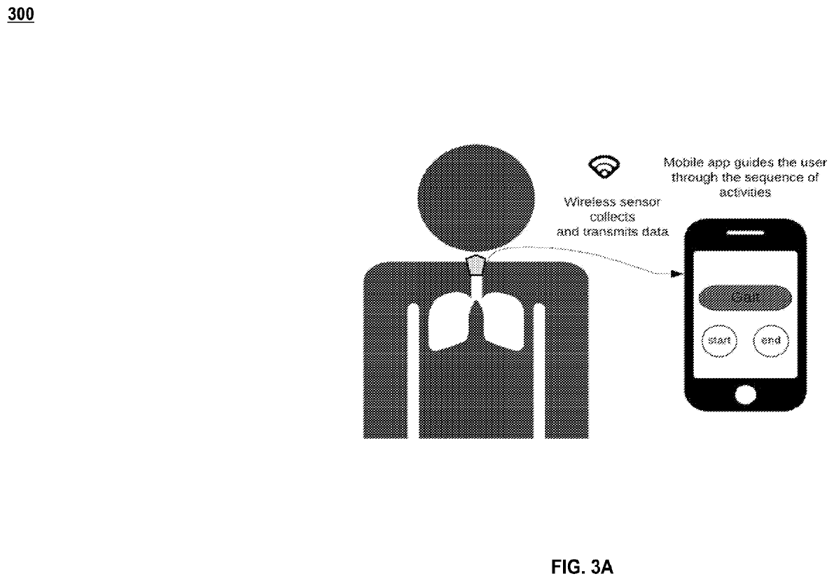

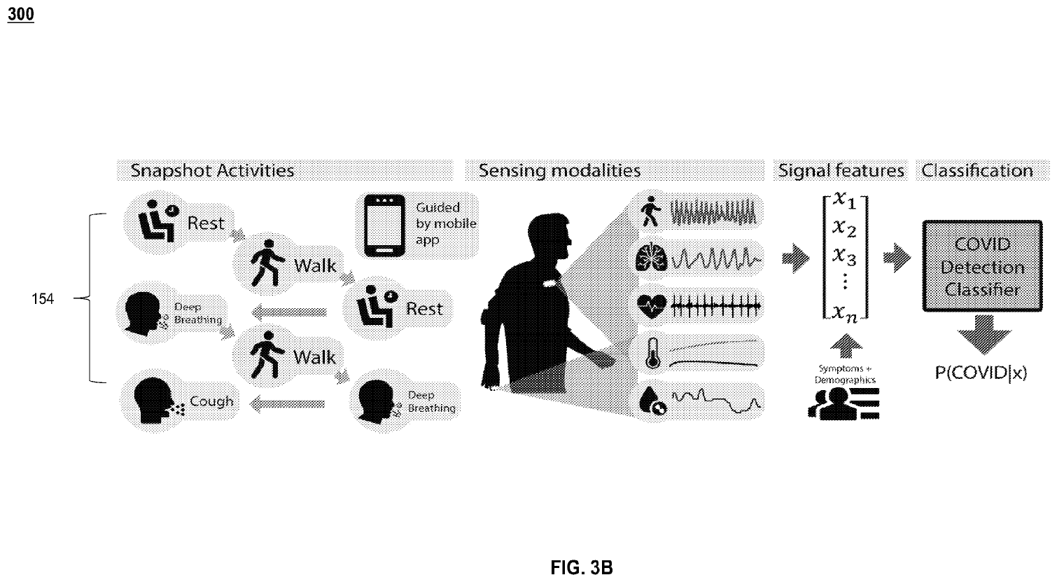

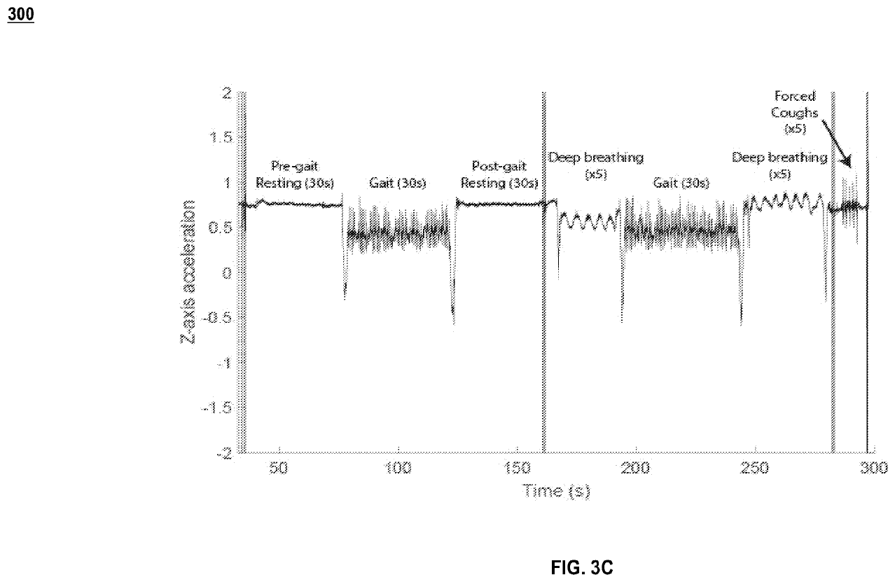

In some examples, as indicated in FIG. 3A, the individual 152 may be prompted by an application executed or otherwise provided by a mobile device to start and stop execution of each activity of the predetermined sequence of activities 154. For example, the individual 152 may be prompted by the application to perform a forced cough five times, and to press a button to start and end the data collection for that activity. After an activity is performed, the data is stored for further processing. A similar process may be repeated for other activities in the predetermined sequence of activities 154. FIGS. 3B-3C show an embodiment of the subject process, along with the raw acceleration signal collected by the movement over time. The complete set of recorded activities is used to uncover the physiological responses associated with disorders or infections such as COVID-19, which includes changes in cardiac, respiratory, and gait patterns.

In some examples, the data (the raw signal from the accelerometer and other sensor modalities) recorded by the sensor system 104 during the execution of each activity of the predetermined sequence of activities 154 is then uploaded wirelessly to a paired mobile device (156 in FIG. 1B) and a cloud server. Sensor data from each activity is processed to extract the relevant physiological signals, which include, but are not limited to: respiration rate and dynamics, heart rate, heart beat dynamics, oxygen saturation, gait and breathing sounds, as indicated in Table 1. These specific physiological signals are extracted using any number of digital filtering and other signal processing techniques. In other embodiments, an activity related to sleep may be included in the predetermined sequence of activities 154. A set of features that are either computed or derived from these signals may include, but is not limited to, physiological characteristics and statistical properties in the time and frequency domains for each recorded activity of the predetermined sequence of activities 154.

TABLE 1
Example Physiological Signals

| Activity | Physiological Signals/Measures |
|---|---|
| Pre-gait | Respiration/Heart Beat Dynamics: Baseline values |
| Rest | |
| Gait | Physical Activity/Fatigue: Energy of walking, Gait frequency, Gait |
| | variability |
| | Respiration/Heart Beat dynamics: Increase relative to baseline |
| Post-gait | Respiration/Heart Beat Dynamics: Increase relative to baseline and |
| Rest | exertion of gait, rate of recovery |
| Deep | Lung Physiology/Heart Beat Dynamics: Inspiration volume, Lung |
| Breathing | sounds |
| Deep | Lung Physiology/Heart Beat Dynamics: Inspiration volume, Lung |
| Breathing | sounds (Increase relative to baseline and exertion of gait, rate of |
| (Post- | recovery) |
| Cough/ | Lung Physiology: Congestion (wet/dry cough), Lung/Airway sounds |
| Talking | |

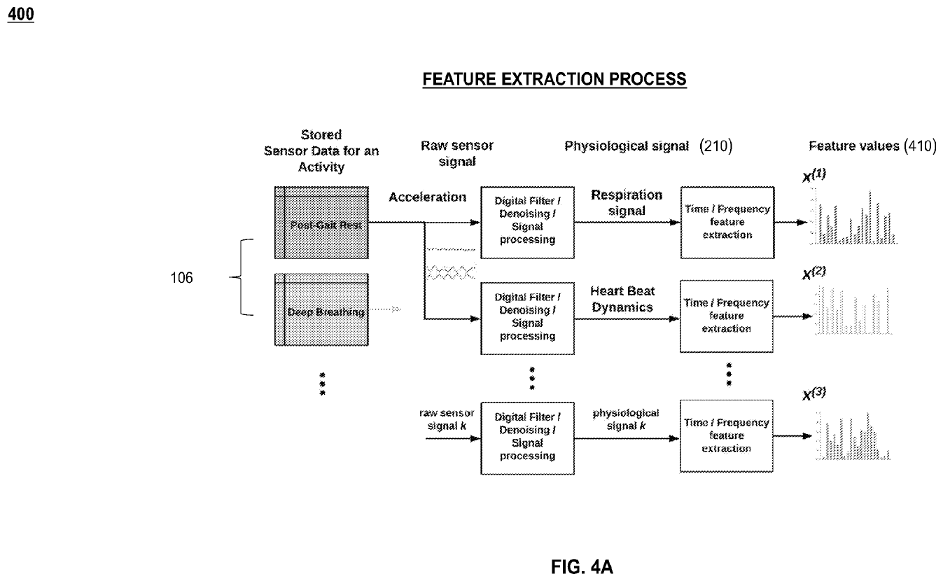

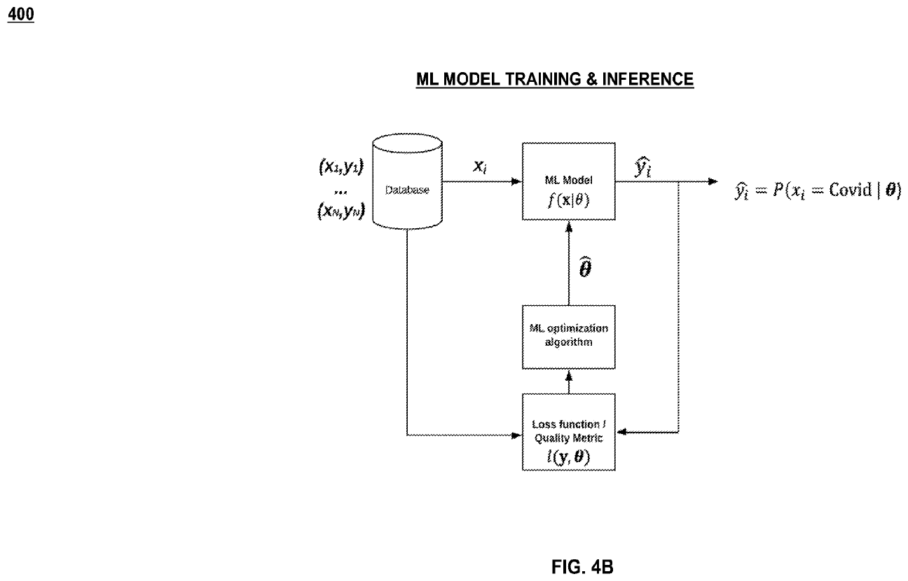

Referring to FIGS. 4A-4B, an exemplary process of configuring or training the model 101 by extracting physiological signals from raw sensor data generated by the sensor system 104 as an individual (for testing or screening) performs the predetermined sequence of activities 154 shall now be described. The subject figures and process refer to a specific example of configuring the model 101 for predicting a positive COVID-19 diagnosis; however, as indicated herein, the steps of deriving physiological signals collectively indicative of a symptom of COVID-19 from raw sensor data or signals generated by the sensor systems 104, and leveraging the physiological signals to optimize the model 101 by extracting feature values from the physiological signals for application to parameters of the model 101 (or otherwise leveraging the physiological signals to customize or configure the model 101), are not limited to COVID-19. As described herein, the functionality and features conveyed by the subject figures may similarly be applied to customize or configure the model 101 to predict a diagnosis of any disorder or symptom.

By non-limiting example with respect to configuration of the model 101 to diagnose COVID-19, the present disclosure considers that COVID-19 leads to changes in normal heart rate dynamics such that heart rate dynamics and changes thereof are considered to be ideal physiological signals for configuring the model 101 to diagnose COVID-19. Therefore feature values (410 in FIG. 4A) associated with features corresponding to signals described herein that capture heart rate dynamics are extracted from heart rate dynamics signals of the sensor data 106. The typical heart signal is characterized by five salient points denoted as P,Q,R,S,T. The raw sensor signal is digitally filtered to remove noise and motion artifacts. Then various inter beat metrics between the salient points (P,Q,R,S,T) are computed to capture heart dynamics. Some embodiments of these inter beat features can include, but are not limited to, time domain, frequency domain, spatial domains, signal statistical properties, derived non-dimensional metrics and composite combination of these features that provide medically relevant inference for predicting COVID. In one such embodiment, various orders of statistical properties including, but not limited to, mean, median, mode, variance, range, quartiles, standard deviation, skewness, kurtosis, coefficient of variation, first and higher order moments, entropy of the inter beat RR interval (i.e. the time difference between the R point in two consecutive heart beats computed over the activities recorded) along with heart rate (beats per minute), and change of heart rate between the sequence of activities are extracted as features to be input to the machine learning/AI model (101).

In a similar fashion, respiration rate and lung function is known to be affected by COVID, such that respiration rate and lung functions signals of the sensor data 106 are considered to be ideal physiological signals for configuring the model 101 to diagnose COVID-19. Therefore, features that capture the respiration signal are extracted from the acceleration signal during different activities using digital filtering techniques to remove noise and motion artifacts, and isolate the breathing or cough signal. In one example, a combination of features in time and frequency domain is used to capture cough, respiration rate and their dynamics. These features include, but are not limited to, the ones mentioned above, and the following: crest factor, spectral skewness, spectral centroid, spectral spread and spectral entropy. Signal differences and quotients relative to their pre-activity baseline are also included.

In addition, gait patterns and movement features relating to physical effort may be indicative of COVID and correlated with the above. In one embodiment for the gait activity, the raw accelerometer data is converted into the frequency domain. As above, statistical features including, but not limited to, mean, standard deviation, range, IQR, variance, kurtosis, skewness, root mean square or sample entropy are extracted and used as features.

In some examples, additional data may optionally be leveraged to further tailor or configure the model 101. For example, demographic features such as age, gender, height, weight, BMI, smoking status and medical history (presence of specific symptoms and onset, medications, existing conditions/co-morbidities, vaccination status) may also be used as inputs to the model 101.

All feature values 410 are aggregated across signals and activities of the predetermined sequence of activities 154, and used as the input to a ML/AI model 101, which has been trained to predict either the presence/absence of respiratory infection, or the risk of having contracted the infection. In some examples, the model 101 combines aspects of the sensor data 106 with available medical history to act as an expert decision system to provide a risk prediction for the existence or absence of COVID or other cardio-respiratory illness. The model 101 may be developed on the basis of a large pool of data collected from individuals with either 1) no known current illness (healthy) or 2) acute-onset symptoms that could be attributed to COVID-19 (COVID-potential). One of many potential machine learning algorithms, such as logistic regression, support vector machines, decision trees, ensemble methods, or neural networks (deep learning), may be used as the base predictor function of the model 101 or to model generally the statistical associations between the extracted signal features and participant type (healthy vs. COVID-potential). A statistical model may be developed based on data from individuals with known illness status, with the objective of maximizing accurate assessment of illness status (yᵢ) based only on a series of input features taken from wearable signals and medical history (xᵢ) (FIGS. 4A-4B). In either case, the model (101) parameters ( ) are optimized to maximize the probability of detecting the illness status.

Once the model is fit (trained) to the data using a known dataset in this way, the model may be used to generate estimates of risk of active illness, given the same set of extracted features and medical history, from an individual of unknown illness risk. The trained model can be deployed to provide a user with a probability measure of the following equation format:

ŷᵢ=P(xᵢ=Covid|θ),

where yᵢ defines an infection risk for COVID-19.

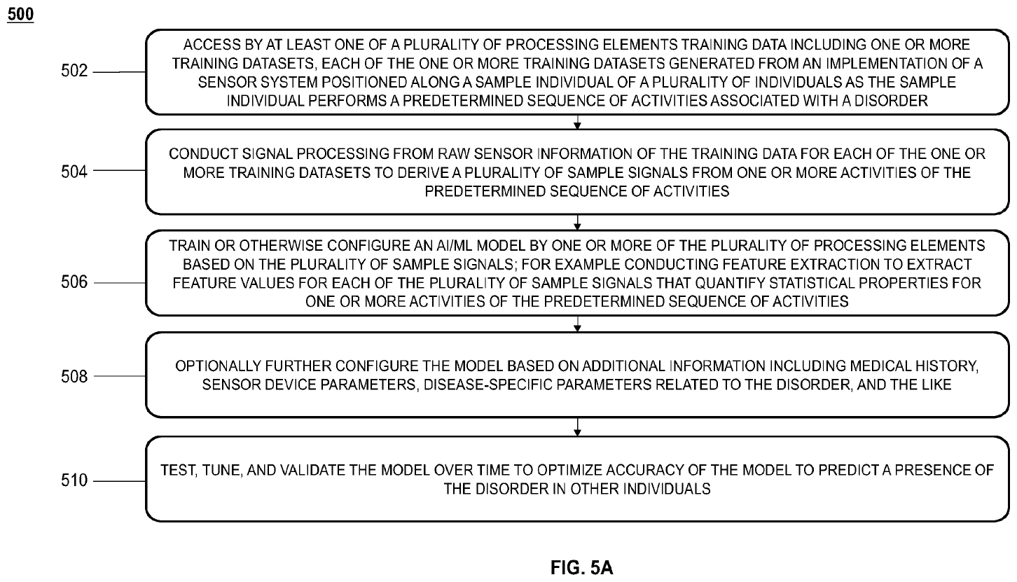

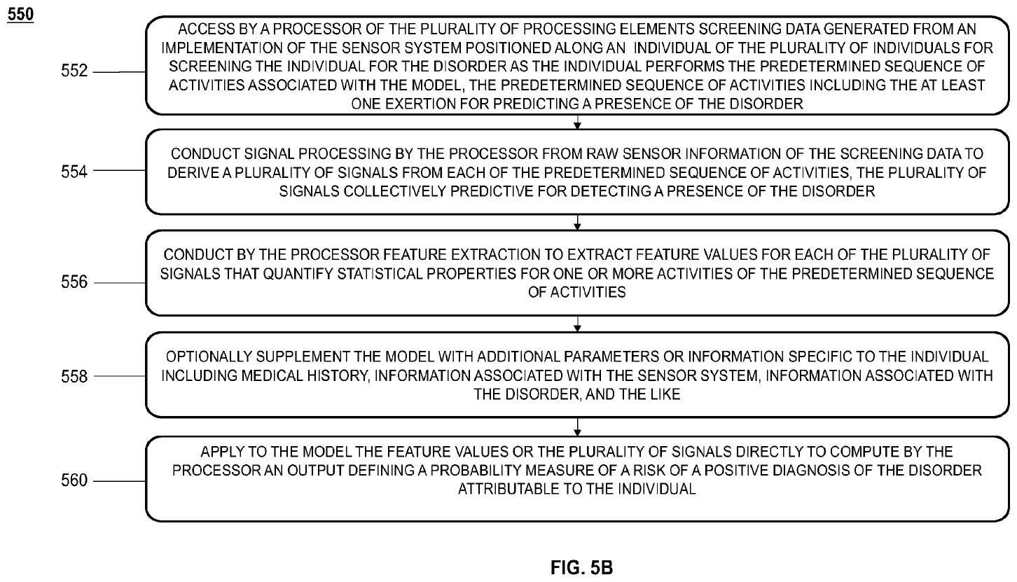

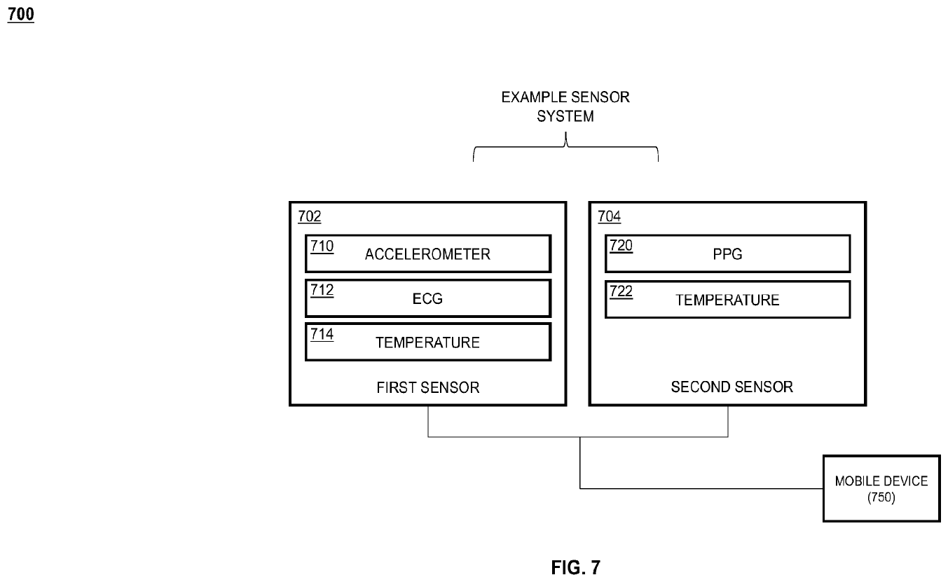

Referring to FIGS. 5A-5B, an example process 500 is shown for configuring/optimizing the model 101 to predict a presence of a disorder, and an example process 550 is further shown for applying the model 101 by the processor 102 to diagnose an individual who needs to be screened for the disorder including but not limited to COVID-19 and infections by other viral pathogens, bacterial pathogens, asthma, and COPD. As indicated by block 502 of the process 500, training datasets are first acquired by some processing element of a plurality of processing elements. Referring back to FIG. 1A, the processor 102, the cloud devices 114, and other computing devices such as the client device 110 all collectively represent a plurality of possible processing elements. As such, the training, configuration, and optimization of the model 101 described by process 500 may be conducted by any one of the subject processing elements or combinations thereof. As further indicated in block 502, the training datasets are generated from an implementation of the sensor system 104 positioned along a sample individual of a plurality of individuals as the sample individual performs a predetermined sequence of activities 154, such as the example predetermined sequence of activities illustrated in FIG. 3B. An exemplary, non-limiting embodiment 700 of the sensor system 104 is shown in FIG. 7. As indicated, the embodiment 700 of the sensor system 104 may include a first sensor 702 which may be positioned along a chest of an individual, and a second sensor 704 which may be positioned along a finger of the individual, each equipped with a plurality of sensor devices. For example, the first sensor 702 may include an accelerometer 710, an electrocardiogram (ECG) sensing device 712, and a temperature sensing device 714. The accelerometer 710 may be a tri-axis accelerometer used to compute broad body movements such as gait patterns, as well as small chest movements due to respiration and cough/deep breathing lung sounds, and the ECG sensing device 712 records heart dynamics such as heart rate, and heart rate variability. The second sensor 704 may include a photoplethysmogram (PPG) device 720 for measuring blood oxygen levels, and a temperature sensing device 722. A mobile device 750 may be in operable communication with the embodiment 700 of the sensor system to prompt the user to perform an activity or otherwise interact with the user to acquire signal data for configuration and application of the model 101 as described herein. The embodiment 700 of the sensor system 104 is suitable for acquiring sensor data defining a plurality of signals helpful for diagnosing COVID-19, in view of the predetermined sequence of activities 154 shown in FIG. 3B. However, it should be understood that variations to the embodiment 700 of the sensor system 104 are contemplated for different disorders, and associated sequence of activities. For example, additional sensing devices may be implemented given the introduction of different activities to the sequence of activities.

Referring to blocks 504 and 506, once the training datasets are acquired by some processing element, the processing element conducts signal processing from the raw sensor information of the training data for each of the one or more training datasets to derive a plurality of sample signals from each of the one or more activities of the predetermined sequence of activities. The plurality of signals are then used to train or otherwise configure the model 101. More specifically, in some examples, a processing element of the plurality of processing elements conducts feature extraction to extract feature values for each of the plurality of sample signals that quantify statistical properties for one or more activities of the predetermined sequence of activities. Alternatively, the sample signals may simply be fed to the model 101 directly without feature extraction (e.g., where model 101 includes a neural network or deep learning). In some examples, the processing element aggregates multiple features across a portion of the plurality of sample signals for a portion of the predetermined sequence of activities, and applies the feature values as inputs to the model 101. Feature values may relate to averages, standard deviations, ranges, minimums, maximums, root-mean squared, quantiles, moments, entropy metrics, skewness, kurtosis, and linear and non-linear metrics. Feature values may further relate to frequency domain features including power spectral density features, peak frequency, power skewness, kurtosis, entropy, center, and spread.

As indicated in block 508, the model 101 may optionally be further configured or trained using additional information including medical history specific to the individual being sampled or like individuals, sensor device parameters or characteristics, and disorder-specific information. Variations to the model 101 may be effectuated to, e.g., account for different environments, types of individuals, and the like.

Referring to block 510, the model 101 may then be tested, tuned and validated over time to optimize the ability of the model 101 to accurately predict a presence of the disorder in other individuals. For example, the model 101 may be trained using additional training data in a similar fashion until the model 101 accurately predicts the presence of the disorder in subsequent sensor data according to a predefined accuracy threshold. In either case, the model 101 as trained and optimized is then suitable for deployment for individuals that need to be screened. In some examples, the model 101 as trained outputs a probability measure P(x=Disorder( )) of infection or affliction risk of the disorder. In some examples, the parameters of the model 101 as trained maximize the probability of detecting the disorder. The trained model 101 in the present example can be deployed to provide a user with a probability measure of the following equation format:

ŷᵢP(xᵢ=DISORDER|θ)

where yᵢ defines an infection risk for the disorder.

Referring to block 552 of process 550, the model 101 as trained is then applicable to individuals that need to be screened or monitored for the disorder. As indicated in block 552, some processor or processing element, such as the same processing element used to train the model 101, a different processing element, or a combination of the same, accesses screening data generated by an implementation of the sensor system 104 as an individual that needs to be screened performs the same predetermined sequence of activities 154 utilized during training. As with the training, the predetermined sequence of activities includes at least one exertion by the individual; i.e., some form of movement, motion, or change in state of the individual that induces a physiological change. By non-limiting examples, an exertion includes jumping, walking, coughing, breathing, and the like. In some embodiments the processing element applying the model 101 is a mobile device or client device.

Referring to block 554, block 556, and block 558, the processor applying the model 101 conducts signal processing from raw sensor data generated from the sensor system 104 to derive a plurality of signals from each of the predetermined sequence of activities. The plurality of signals collectively are predictive for detecting the presence of the disorder. In some examples, the processor further conducts feature extraction to extract feature values for each of the plurality of signals that quantify statistical properties for one or more activities of the predetermined sequence of activities. Optionally, the model 101 may be tuned or configured for the individual being screened prior to application by supplementing the model with additional information such as medical history specific to the individual being sampled or like individuals, sensor device parameters or characteristics, and disorder-specific information.

Referring to block 560, aspects of the plurality of signals derived from the sensor data generated are applied to the model 101 to compute an output. In some examples, the output includes a probability measure of a risk of a positive diagnosis of the disorder attributable to the individual. Where features are extracted from the plurality of signals, values associated with the features as extracted are fed to the model 101 to compute the output (FIG. 4B). Alternatively, signals may be directly applied to the model 101. In some examples, the model 101 is a probabilistic model such that the output defines a number between 0 and 1, wherein 0 predicts a minimal probability of a positive diagnosis of the disorder by the individual being screened.

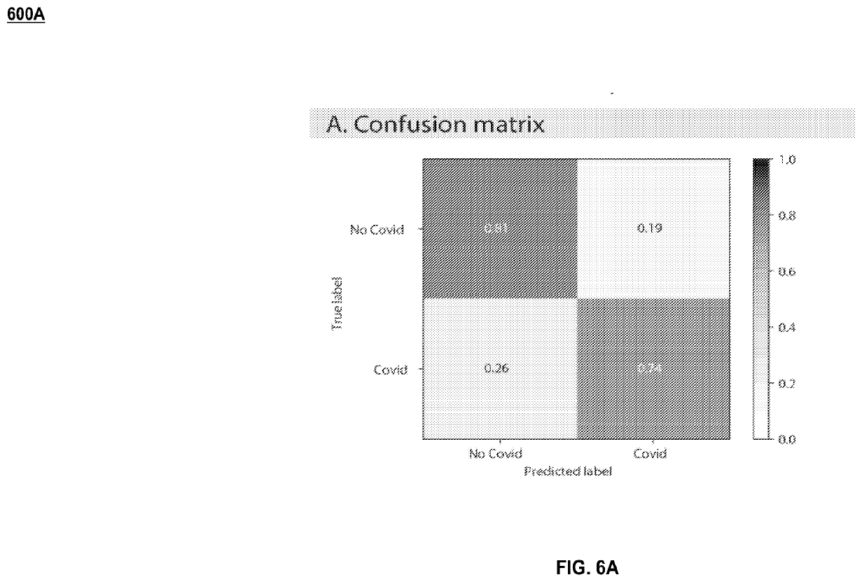

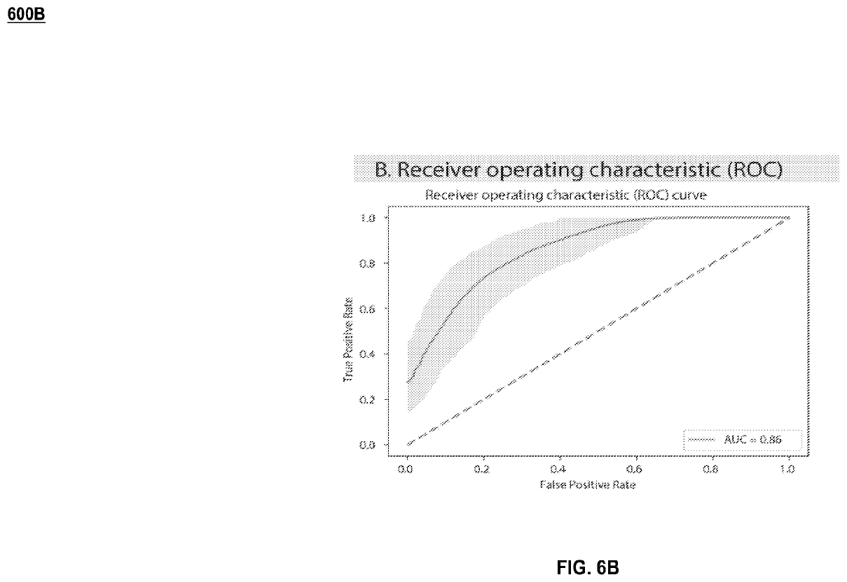

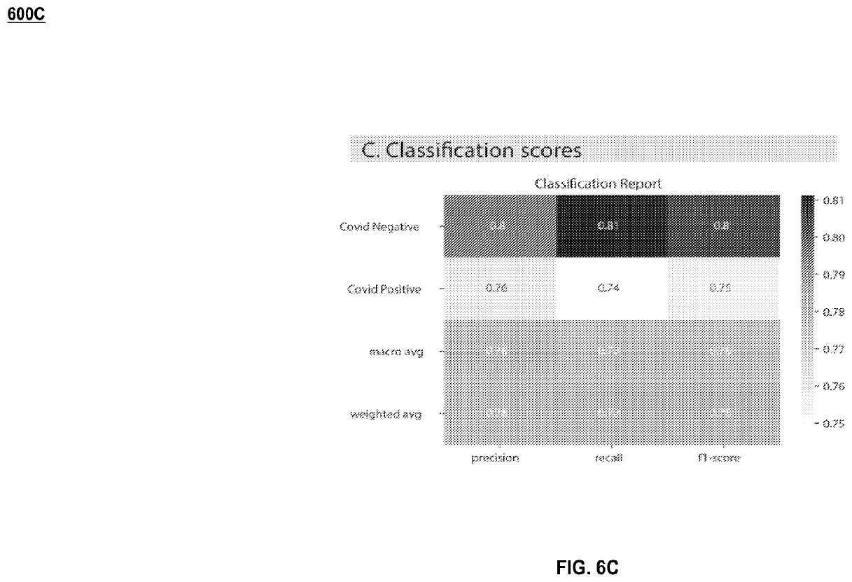

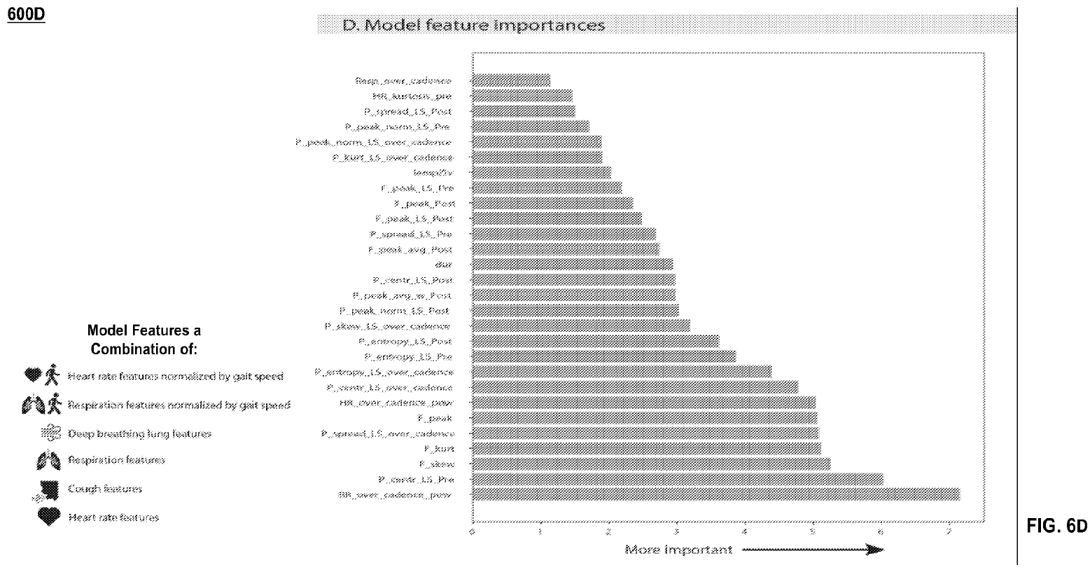

Referring to FIGS. 6A-6D, testing data illustrating the efficacy of the model 101 is illustrated. Specifically, an example of the model 101 was applied to testing participants for COVID-19. Sensor data using the sensor systems 104 was collected from 96 participants in India: 43 COVID positive patients, and 53 COVID negative healthy controls. Each participant was given a chest and finger sensor and performed the following exemplary predetermined sequence of activities (154): rest (30 seconds), walk (30 seconds), rest (30 seconds), deep breaths (×5), walk (30 seconds), deep breaths (×5), and forced coughs (×5). From the chest sensor, cardiac activity was computed using the ECG, respiration rate from the accelerometer, and gait dynamics using the accelerometer. From the finger sensor, temperature and 402 values were measured. From these physiological signals, a set of time domain and frequency domain features were computed that correspond to each event in the predetermined sequence of activities (154).

Using leave-one-subject-out cross validation, an algorithm associated with a machine learning classifier (Random Forest Classifier) was selected as a basis for the model 101 to differentiate between participants who had COVID+ and COVID− PCR tests. The confusion matrix is shown in FIG. 6A, which indicates that the classifier correctly identified 81% of the COVID− participants and 74% of the COVID+ participants. This led to an area under the receiver operating characteristic (ROC) of 0.86 (FIG. 6B), which shows strong classifier performance (a perfect classifier has an area under ROC of 1, random guessing is 0.5). FIG. 6C shows the classification scores with high sensitivity and precision for both classes. Lastly, FIG. 6D shows the features that were used to configure the model 101 to differentiate the COVID positive and negative participants. A wide range of features were used in training the algorithm, which include heart rate, respiration rate, deep breathing lung sounds, and gait dynamics. As more data flows in from the subject India deployment, the algorithm may be further tuned and validated.

In some examples, the signal processing conducted during the training and configuration process 500 and the application process 550 is conducted post-processing; meaning after the recordation of the sensor data 106. However, other examples and variations encompass real-time signal processing of at least some of the signals at each stage.

### Exemplary Computing Device

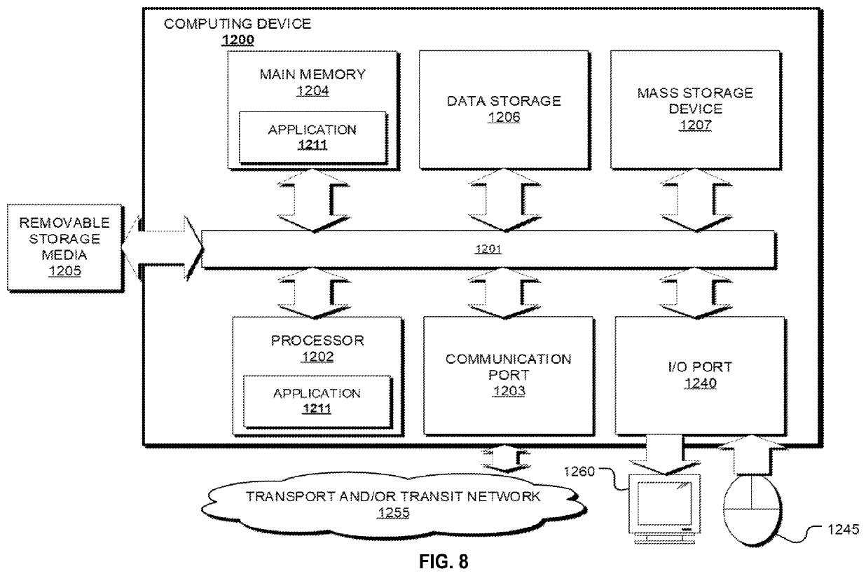

Referring to FIG. 8, a computing device 1200 is illustrated which may be one of the processing elements described herein and/or the place of the processor 102 and be configured, via one or more of an application 1211 or computer-executable instructions, to execute functionality described herein. More particularly, in some embodiments, aspects of the predictive methods herein may be translated to software or machine-level code, which may be installed to and/or executed by the computing device 1200 such that the computing device 1200 is configured to execute functionality described herein. It is contemplated that the computing device 1200 may include any number of devices, such as personal computers, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments, and the like.

The computing device 1200 may include various hardware components, such as a processor 1202, a main memory 1204 (e.g., a system memory), and a system bus 1201 that couples various components of the computing device 1200 to the processor 1202. The system bus 1201 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computing device 1200 may further include a variety of memory devices and computer-readable media 1207 that includes removable/non-removable media and volatile/nonvolatile media and/or tangible media, but excludes transitory propagated signals. Computer-readable media 1207 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the computing device 1200. Communication media includes computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof. Computer-readable media may be embodied as a computer program product, such as software stored on computer storage media.

The main memory 1204 includes computer storage media in the form of volatile/nonvolatile memory such as read only memory (ROM) and random access memory (RAM). A basic input/output system (BIOS), containing the basic routines that help to transfer information between elements within the computing device 1200 (e.g., during start-up) is typically stored in ROM. RAM typically contains data and/or program modules that are immediately accessible to and/or presently being operated on by processor 1202. Further, data storage 1206 in the form of Read-Only Memory (ROM) or otherwise may store an operating system, application programs, and other program modules and program data.

The data storage 1206 may also include other removable/non-removable, volatile/nonvolatile computer storage media. For example, the data storage 1206 may be: a hard disk drive that reads from or writes to non-removable, nonvolatile magnetic media; a magnetic disk drive that reads from or writes to a removable, nonvolatile magnetic disk; a solid state drive; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media provide storage of computer-readable instructions, data structures, program modules, and other data for the computing device 1200.

A user may enter commands and information through a user interface 1240 (displayed via a monitor 1260) by engaging input devices 1245 such as a tablet, electronic digitizer, a microphone, keyboard, and/or pointing device, commonly referred to as mouse, trackball or touch pad. Other input devices 1245 may include a joystick, game pad, satellite dish, scanner, or the like. Additionally, voice inputs, gesture inputs (e.g., via hands or fingers), or other natural user input methods may also be used with the appropriate input devices, such as a microphone, camera, tablet, touch pad, glove, or other sensor. These and other input devices 1245 are in operative connection to the processor 1202 and may be coupled to the system bus 1201, but may be connected by other interface and bus structures, such as a parallel port, game port or a universal serial bus (USB). The monitor 1260 or other type of display device may also be connected to the system bus 1201. The monitor 1260 may also be integrated with a touch-screen panel or the like.

The computing device 1200 may be implemented in a networked or cloud-computing environment using logical connections of a network interface 1203 to one or more remote devices, such as a remote computer. The remote computer may be a personal computer, a server, a router, a network PC, a peer device or other common network node, and typically includes many or all of the elements described above relative to the computing device 1200. The logical connection may include one or more local area networks (LAN) and one or more wide area networks (WAN), but may also include other networks. Such networking environments are commonplace in offices, enterprise-wide computer networks, intranets and the Internet.

When used in a networked or cloud-computing environment, the computing device 1200 may be connected to a public and/or private network through the network interface 1203. In such embodiments, a modem or other means for establishing communications over the network is connected to the system bus 1201 via the network interface 1203 or other appropriate mechanism. A wireless networking component including an interface and antenna may be coupled through a suitable device such as an access point or peer computer to a network. In a networked environment, program modules depicted relative to the computing device 1200, or portions thereof, may be stored in the remote memory storage device.

Certain embodiments may be described herein as including one or more modules. Such modules are hardware-implemented, and thus include at least one tangible unit capable of performing certain operations and may be configured or arranged in a certain manner. For example, a hardware-implemented module may comprise dedicated circuitry that is permanently configured (e.g., as a special-purpose processor, such as a field-programmable gate array (FPGA) or an application-specific integrated circuit (ASIC)) to perform certain operations. A hardware-implemented module may also comprise programmable circuitry (e.g., as encompassed within a general-purpose processor or other programmable processor) that is temporarily configured by software or firmware to perform certain operations. In some example embodiments, one or more computer systems (e.g., a standalone system, a client and/or server computer system, or a peer-to-peer computer system) or one or more processors may be configured by software (e.g., an application or application portion) as a hardware-implemented module that operates to perform certain operations as described herein.

Accordingly, the term “hardware-implemented module” encompasses a tangible entity, be that an entity that is physically constructed, permanently configured (e.g., hardwired), or temporarily configured (e.g., programmed) to operate in a certain manner and/or to perform certain operations described herein. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor may be configured as respective different hardware-implemented modules at different times. Software may accordingly configure the processor 1202, for example, to constitute a particular hardware-implemented module at one instance of time and to constitute a different hardware-implemented module at a different instance of time.

Hardware-implemented modules may provide information to, and/or receive information from, other hardware-implemented modules. Accordingly, the described hardware-implemented modules may be regarded as being communicatively coupled. Where multiple of such hardware-implemented modules exist contemporaneously, communications may be achieved through signal transmission (e.g., over appropriate circuits and buses) that connect the hardware-implemented modules. In embodiments in which multiple hardware-implemented modules are configured or instantiated at different times, communications between such hardware-implemented modules may be achieved, for example, through the storage and retrieval of information in memory structures to which the multiple hardware-implemented modules have access. For example, one hardware-implemented module may perform an operation, and may store the output of that operation in a memory device to which it is communicatively coupled. A further hardware-implemented module may then, at a later time, access the memory device to retrieve and process the stored output. Hardware-implemented modules may also initiate communications with input or output devices.

Computing systems or devices referenced herein may include desktop computers, laptops, tablets e-readers, personal digital assistants, smartphones, gaming devices, servers, and the like. The computing devices may access computer-readable media that include computer-readable storage media and data transmission media. In some embodiments, the computer-readable storage media are tangible storage devices that do not include a transitory propagating signal. Examples include memory such as primary memory, cache memory, and secondary memory (e.g., DVD) and other storage devices. The computer-readable storage media may have instructions recorded on them or may be encoded with computer-executable instructions or logic that implements aspects of the functionality described herein. The data transmission media may be used for transmitting data via transitory, propagating signals or carrier waves (e.g., electromagnetism) via a wired or wireless connection.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A non-invasive method of predicting a disorder diagnosis that does not require presence of a clinician during operation, comprising:
accessing by a processor of a plurality of processing elements screening data generated from a sensor system positioned along an individual of a plurality of individuals for screening the individual for a disorder as the individual performs a predetermined sequence of activities, the predetermined sequence of activities including at least one exertion configured for predicting a presence of the disorder;
conducting signal processing by the processor from raw sensor information of the screening data to derive a plurality of signals from each activity of the predetermined sequence of activities, the plurality of signals from an activity collectively predictive for detecting the presence of the disorder; and
computing by the processor an output defining a probability measure of risk of a positive diagnosis of the disorder attributable to the individual by applying the plurality of signals defined by the screening data to a machine learning model, parameters of the machine learning model configured, based on the plurality of signals, to maximize a probability of detecting the disorder.

2. The method of claim 1, further comprising:
configuring the machine learning model, by:
accessing by at least one of the plurality of processing elements one or more training datasets, each of the one or more training datasets generated from an implementation of the sensor system positioned along a sample individual of the plurality of individuals as the sample individual performs the predetermined sequence of activities; and
conducting signal processing by the processor for each of the one or more training datasets to derive a plurality of sample signals from one or more activities of the predetermined sequence of activities, wherein the machine learning model is trained and configured based on the plurality of sample signals.

3. The method of claim 2, further comprising training the machine learning model by conducting feature extraction by the processor to extract feature values for each of the plurality of sample signals that quantify statistical properties for one or more activities of the predetermined sequence of activities.

4. The method of claim 3, further comprising:
aggregating by the processor multiple feature values across a portion of the plurality of sample signals for a portion of the predetermined sequence of activities, and applying all of the feature values as inputs to the machine learning model.

5. The method of claim 3, wherein the feature values relate to averages, standard deviations, ranges, minimums, maximums, root-mean squared, quantiles, moments, entropy metrics, skewness, kurtosis, and linear and non-linear metrics.

6. The method of claim 3, wherein the feature values relate to frequency domain features including power spectral density features, peak frequency, power skewness, kurtosis, entropy, center, and spread.

7. The method of claim 1, further comprising detecting by the processor changes to the plurality of signals of the screening data during a pre-exertion activity, during an exertion activity including the at least one exertion, and during a post-exertion activity of the predetermined sequence of activities.

8. The method of claim 1, wherein the machine learning model is a probabilistic model such that the output defines a number between 0 and 1, wherein 0 predicts a minimal probability of a positive diagnosis of the disorder by the individual being screened.

9. The method of claim 1, further comprising:
applying to the machine learning model additional data derived from medical history information associated with the individual being screened or like individuals, diseases specific domain knowledge, or sensor features.

10. The method of claim 2, wherein the plurality of signals and the plurality of sample signals include physiological, motion, and mechano-acoustic signals associated with the symptom of the disorder.

11. The method of claim 1, wherein the at least one exertion of the predetermined sequence of activities includes a predetermined action by the individual that results in a physiological or mechanical change.

12. The method of claim 1, wherein the sensor system includes a first sensor positioned along a chest of the individual to monitor movement and gait patterns, respiratory dynamics, and heart dynamics of the individual, and a second sensor positioned along a finger of the individual including a PPG sensing device.

13. The method of claim 12, wherein the first sensor measures acceleration, ECG, and a first temperature, and the second sensor measures blood-oxygen and a second temperature.

14. The method of claim 1, wherein the sensor system includes a motion sensor defining an accelerometer and a photopletysmography (PPG) sensor, such that the plurality of signals includes mechano-acoustic signals recorded by the accelerometer and blood oxygen levels recorded by the PPG sensor.

15. The method of claim 1, wherein the disorder is a COVID-19 infection, and the plurality of signals includes a heart signal, and a change in heart signal between activities in the predetermined sequence of activities is extracted by the processor as a feature for the machine learning model, and the plurality of signals further includes an acceleration signal indicative of a respiration rate of the individual.
